(19) United States
(12) Patent Application Publication
(10) Pub. No.: US 2010/0016742 A1
(43) Pub. Date: Jan. 21, 2010

(54) System and Method for Monitoring, Measuring, and Addressing Stress

(76) Inventors: Terry L. James, Fairview, TX (US); Grant K. James, Fairview, TX (US)

Correspondence Address: Patent Capital Group, 6119 McCommas Blvd, Dallas, TX, 75214, US

(21) Appl. No.: 12/176,369

(22) Filed: Jul. 19, 2008

(51) Int. Cl. A61B 5/0402

(52) U.S. Cl. 600/509

(57) ABSTRACT

In accordance with one embodiment of the present invention, a method for quantifying stress is presented. The method includes interacting with an end user through a stress module and processing heart rate variance data collected during a stress test. The heart rate variance data may include a coherence attribute. The method further includes transmitting the heart rate variance data over a network and to a specified location. In more specific embodiments, the method includes delivering a numerical score to the end user. The score signifies a stress level for the end user.

### TECHNICAL FIELD OF THE INVENTION

This invention relates in general to health management and, more particularly, to a system and a method for monitoring, measuring, and addressing stress.

### BACKGROUND OF THE INVENTION

‘Stress’ may be fairly characterized as the Epidemic of the 21^{st} Century. Insidious in an isolated form, stress engenders a host of other problems that seem to feed off stress conditions. Numerous recent surveys confirm that adult Americans perceive they are under more stress than a decade or two ago. Job Stress is clearly the leading source of stress for adults, but stress levels have also escalated in children, teenagers, college students, and the elderly for other reasons (e.g., peer pressures, unhealthy life style habits, social isolation and loneliness, the erosion of family and religious values, the loss of other sources of social support that combat stress, etc).

The physical manifestations of high stress levels typically include increased heart rate and blood pressure, increases in blood sugar to furnish more fuel for energy as the result of the breakdown of glycogen, fat, and protein stores, and increased nervous system activity, which generally includes an outpouring of adrenaline, cortisol, and other stress-related hormones. Chronic and more problematic stress due to loneliness, poverty, bereavement, depression, and frustration are associated with impaired immune system resistance to viral linked disorders ranging from the common cold to cancer. Recent research in the stress area has helped to explain how stress can contribute to depression and anxiety and, furthermore, the research outlines how stress can impair the gastrointestinal tract, skin, and other organs.

Numerous companies have embarked on the difficult challenge of managing stress and, further, attempting to both quantify and attenuate stress. For example, HeartMath of Boulder Creek, Calif. (www.heartmath.com) has developed a hand-held device for qualifying stress levels of a person (e.g., High, Medium, and Low stress levels). While current solutions offer some assistance in addressing stress, these strategies and products fail to comprehensively address many prevalent stress issues. One void in this stress management field is the ability to offer the consumer a useful metric that indicates a certain level of stress.

### SUMMARY OF THE INVENTION

From the foregoing, it may be appreciated that a need has arisen for an improved process for achieving superior measuring and monitoring of stress levels. In accordance with the present invention, disadvantages and problems associated with previous techniques for addressing stress issues are overcome.

In accordance with one embodiment of the present invention, a method for quantifying stress is presented. The method includes interacting with an end user through a stress module and processing heart rate variance data collected during a stress test. The heart rate variance data may or may not include a coherence attribute. The method further includes transmitting the heart rate variance data over a network and to a specified location. In more specific embodiments, the method includes delivering a numerical score to the end user. The score signifies a stress level for the end user.

In more specific embodiments, the score is based on a plurality of time intervals associated with the test. The interacting is performed through a website, a health station, or a personal computer. Alternatively, the interacting is performed through a handheld device. The handheld device is a cellular phone, an I-Phone, a PDA, or a handheld stress test tool.

In yet other embodiments, the test includes designations for Low, Medium, and High stress levels, whereby each stress level includes a respective range of stress scores. The test can include sounds that are played for the end user, or messaging provided to the end user. The stress test can also include a color scheme that indicates levels of stress for the end user.

Important technical advantages of certain embodiments of the present invention include providing an effective algorithm that, unlike other systems, gives an end user a tangible metric and, further, details whether that metric is acceptable, poor, good, etc. This provides a necessary context for the end user to better understand his stress levels. Moreover, such a measurement can be retrieved over a network (e.g., the Internet), which currently is not being done. Note that any heart rate variance data can be transmitted (e.g., over a network) and this too is not being done currently. Once the data is obtained, then the manager or administrator of the data can process that information in any chosen manner. For example, an administrator can make comparisons, encourage certain types of activity, discourage other actions, incentivize the end user, etc. Thus, stress quantification may occur in a community setting or a group environment, which empowers the administrator to stratify groups, aggregate scores, etc. Groups could be organized by age, gender, race, blue-collar vs. white-collar, morbidity, income, job description, etc. These abilities, and other advantages, are further detailed and discussed below with reference to the FIGURES.

Other technical advantages of the present invention will be readily apparent to one skilled in the art from the following figures, descriptions, and claims. Moreover, while specific advantages have been enumerated above, various embodiments may include all, some, or none of the enumerated advantages.

### BRIEF DESCRIPTION OF THE DRAWINGS

For a more complete understanding of the present invention and its advantages, reference is now made to the following descriptions, taken in conjunction with the accompanying drawings, in which:

FIG. 1 is a simplified block diagram that illustrates a system for providing a stress measuring tool through interaction with selected devices and with a health station connected to a network in accordance with a particular embodiment of the present invention; and

FIGS. 2-4 are simplified screen shots of how a stress module can operate for one or more end users.

### DETAILED DESCRIPTION OF THE INVENTION

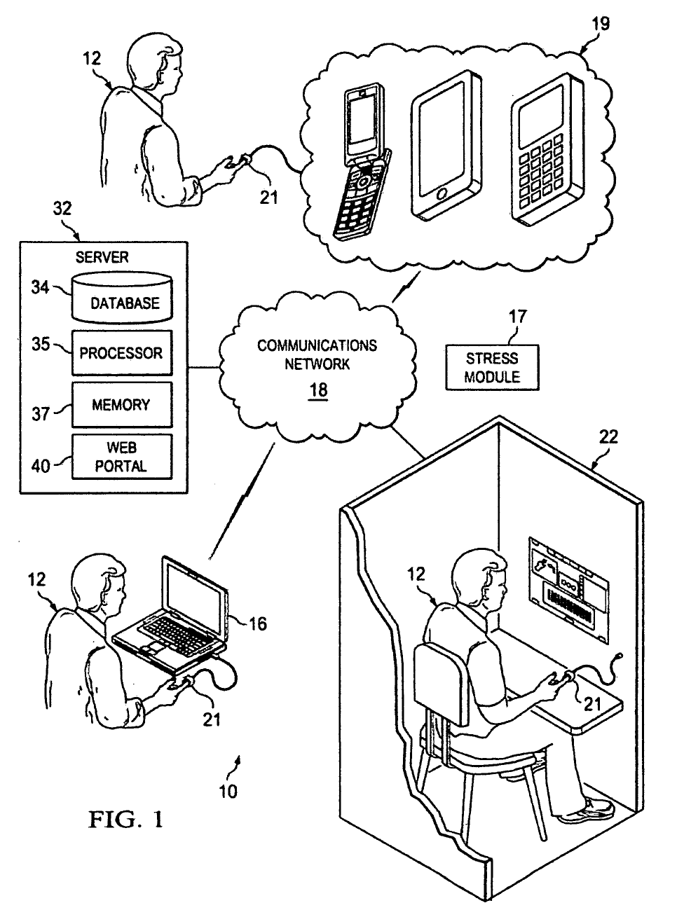

FIG. 1 is a simplified block diagram of a system 10 for providing a stress measuring tool in one example embodiment. In the depicted example, this can be achieved via a health station 22, which is connected to a network. Alternatively, this can be achieved (and is illustrated) through a computer (via a website) or through various handheld devices 19. Handheld devices 19 include a cellular telephone, a portable stress measuring tool, and an I-Phone. Handheld devices 19 may include hardware (including a processor and a memory for storing collected data) and any software application that facilitates the stress measurement, gathering, and storage operations as outlined herein in this document.

In addition, the memory allows measurements to be stored over time and then uploaded to a computer (e.g., PC) and then to a server via the web. In essence, the movement of data [its ability to propagate to various locations] has enormous value. Hence, in accordance with the present invention, any heart rate variance measurement can be sent/retrieved over a network (e.g., the Internet), which currently is not being done.

Furthermore, a unique identifier can be attached to each individual who has a stress test. Hand held devices 19 can similarly have a unique identifier. Some form of log-in could be used with this identifier. For example, the user could log into handheld devices 19 (e.g., a username and password, which could include the unique identifier), or at a health station, or a PC, where the individual could simply log in with a unique identifier.

A stress module 17 is also provided and this module may be included in health station 22, in a personal computer (inclusive of laptops), on a server, on a web site, in the hand-held device, on the cellular telephone, on a Personal Digital Assistant (PDA), on the I-Phone, etc. Each of these components includes a memory and a processor that is operable to execute the functions of stress module 17.

Before turning to some of the components of certain embodiments of the present invention, some preliminary information about a typical data collection protocol is provided. It is beneficial to provide an overview as to the way in which the following invention operates. The following foundational information may be viewed as a basis from which the present invention may be properly explained. Such information is offered earnestly for purposes of explanation only and, accordingly, should not be construed in any way to limit the broad scope of the present invention and its potential applications.

Typically, stress measurement techniques focus on heart rate variability (HRV), which can be tied to the nervous system. These bio-feedback parameters are not necessarily addressing all the issues being presented by stress. HRV is a measure of the beat-to-beat variations in heart rate. HRV is usually calculated by analyzing a series of beat-to-beat intervals from an electrocardiogram (ECG) or of beat-to-beat intervals derived from an arterial pressure tracing. HRV is regarded as an indicator of the activity of autonomic regulation of circulatory function.

In accordance with the teachings of the present invention, an algorithm is used to quantify stress by assigning a numeric value to the given level of stress. In addition, the end user is being given a specific number with an accompanying range of stress levels. Thus, the algorithm of the present invention, unlike other systems, actually gives an end user a tangible metric and, further, details whether that metric is acceptable, poor, good, etc. This provides a necessary context for the end user to better understand his stress levels. Moreover, such a measurement can be sent/relayed/retrieved over a network (e.g., the Internet), which currently is not being done by other architectures. Stress module 17, potentially in cooperation with any of the devices of FIG. 1, can measure HRV data and then upload that information to the Internet, a server, a web-site, etc. Thus, stress module can facilitate any type of suitable web-service.

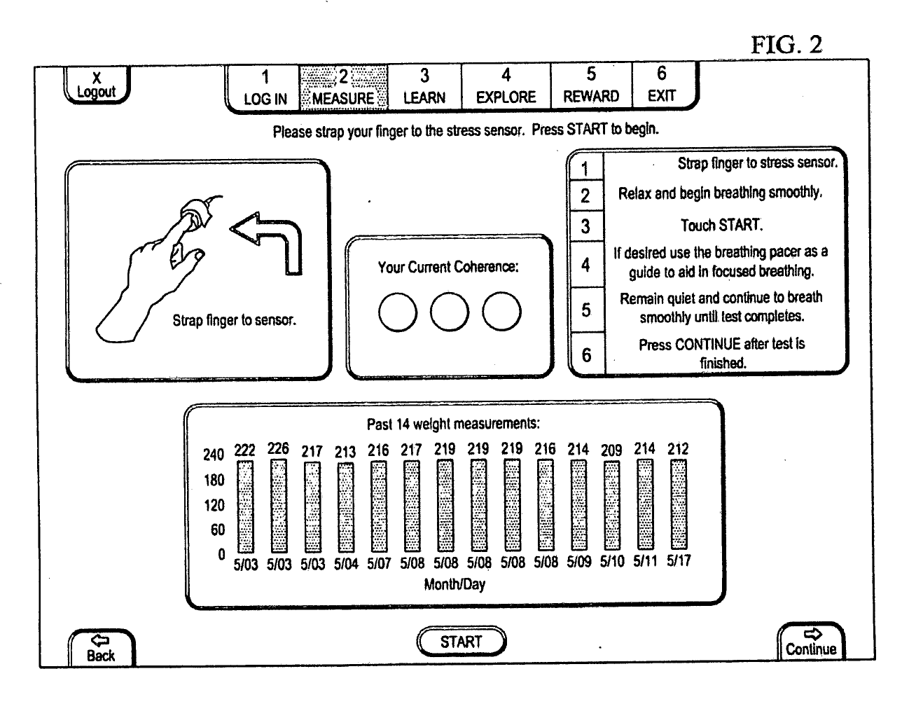

Once the data is obtained, then the manager or administrator of the data can process that information in any chosen manner. For example, an administrator can make comparisons, encourage certain types of activity, discourage other actions, incentivize the end user, etc. Through a network-based approach, stress quantification may occur in a community setting or a group environment, which empowers the administrator to stratify groups, aggregate numerical scores, etc. Groups could be organized by age, gender, race, blue-collar vs. white-collar, morbidity, income, job description, etc. These abilities are further detailed and discussed below with reference to FIGS. 2-4.

Note that health station 22 can also serve as a single point of collection for various metrics (as described further below). The stress measurement component can be part of a complete data collection process. Thus, health station 22 can measure stress for a single end user (or a group of end users) and subsequently provide follow-up teaching modules that inform the end user about possible causes of stress, ways to combat stress, etc. The applications and software modules within health station 22 (and the on-line community website) can readily interact with this stress component. In one example, each member of a community or organization would visit health station 22 once per month and be accessed for stress (through questionnaires and physically measuring biometrics, stress levels, etc.) and then receive training about minimizing stress. This could include video tutorials, interactive games, biofeedback, pamphlets, etc. One option may be provided (through a web site, through health station 22, etc.) to purchase devices that further monitor stress, or purchase educational tools that would educate users about the importance of stress management. These instructional opportunities and possible designs are further detailed below.

The administrator can also help to identify sources of stress, possible stress triggers (financial, relationship, employment), and formulate some treatment approaches for these specific issues. Moreover, the administrator can offer suitable content to the end user (or group of end users) to help attenuate some of these stress issues, as well as better educate this population about how to better deal with these stress issues. Also, the administrator can directly address the psychological issues being presented by these stress issues.

There are certain inherent advantages to having a single point of collection (for example, through a network-based approach, or any combination of technologies that include health station 22). This is because a single entity (for example, the administrator) can see all the physical data for a single person or a group, and then treat the target population based on all the symptoms or signals being provided by the patient pool. In many cases, only understanding one factor (for example high blood pressure) may not confer the requisite knowledge for a tending physician to make a valid prognosis, or to offer an effective treatment. If the knowledge of ‘high blood pressure’ is then coupled with information concerning stress levels (for example, this particular end user is expecting to be laid off shortly), then that combination of factors would empower a physician with a more complete picture of this patient's circumstances.

In another example, which is illustrative, a group of end users may be revealing an unusually high incidence of stress. A simple comparison of these users may indicate that everyone exhibiting these high stress rates is working the ‘night shift’ at a given company. Lack of sleep does lead to increased appetites, which may also help demonstrate why this particular group is obese, which may further in turn reveal why the incidence of diabetes is so high in this particular group. Again, there is tremendous power in having all of this important information in a single location and this leads to better treatments for the end user or group of end users.

Turning back now to the depicted infrastructure of FIG. 1, FIG. 1 represents a streamlined version of system 10 for collecting, displaying, converting, and processing stress data in a network environment. System 10 includes a communications network 18, one or more end users 12, one or more computer devices 16, one or more stress modules 17, one or more servers 32, one or more databases 34, and a web portal 40. Each server can include a processor 35 and a memory 37 that can cooperate to perform any of the various functions as outlined herein: some of which include executing software.

FIG. 1 could also include various other potential components to be used with stress module 17, such as a Wi-Fi network, a cellular network, a Bluetooth connectivity component, a group of satellites, etc. Essentially, anything that can transfer data could be included in such a model.

This example is also provided to show the options a given end user has in receiving his stress data. The end user can use his personal/work computer or health station 22, as is illustrated. It should be noted that the present invention is certainly amenable to be used with non-web-based applications. While there are discussions included herein about uploading information to a server of some sort, the architecture of the present invention (and stress module 17 specifically) can be used in conjunction with servers that are not necessarily web-based. The simplest form of such a server arrangement would be stress module 17 connected to a computer that stores the information itself without uploading it to a web-server. Thus, the present invention can be used in conjunction with both web-based and non-web-based applications alike.

In operation, these elements can cooperate to create a numerical score that is based on heart rate variance. The numerical score effectively and consistently quantifies the stress level of an individual. No such numerical score is provided by current architectures. Additionally, lacking in any other system is the connection from a handheld device (or a personal computer) to a network. For example, the present invention can readily collect heart rate variance data (from a device or from a personal computer) and then send data from that element [via a network] to a server (such as web servers 32).

Note that both a handheld device, as well as a personal computer, is afforded the ability to transmit collected heart rate variance data over the web/network and to a server. In this sense, heart rate variance is measured and then moved from the point of collection to the web and then potentially to a server (e.g., a website). Quantifying the data and yielding a numerical score is valuable, but so too is the notion of capturing the data and then moving the data from the initial point of collection (e.g. the sensor) to a personal computer (or hand-held device), then on to the web, and then potentially to an ultimate server. Once the data has reached the server, then the stress score can be correlated to various disease states. For example, individuals who have high stress scores and also have coronary artery disease would be at hither risk for a heart attack. Moreover, in example embodiments, there can be varying degrees of difficulty that can be deployed for training. Each training level would have a stress score attached to a corresponding degree of difficulty. These operations are further detailed below.

Turning back to the infrastructure depicted in FIG. 1, in general, users 12 can use stress module 17 to track ore or more primary metrics. This could include wearing a portable device, such as that which is depicted in handheld devices 19 cloud of FIG. 1. Users 12 can couple stress module 17 to one or more computer devices 16, which provide users access to a web portal 40. Stress module 17 can transmit data to web portal 40 or receive data from the web portal 40 (two-way communication).

Stress module 17 could also communicate via “piggybacking.” The device would be able to receive data packets from other devices (such as cell phones equipped to take a stress measurement). Stress module 17 can easily be connected to a computer to take stress measurements or to facilitate a connection between the device and a website, for example.

Turning to the website component of stress module 17, system 10 also achieves an effective way for users 12 to view stress data. Web portal 40 allows users 12 to input customized data, such that the data is unique to each user 12. Web portal 40 is operable to process this customized data. Stress module 17 is operable to monitor, calculate, and display user's stress data in a format selected by user 12. Stress module 17 can display a user's current level of stress performance, or stress module 17 can continually update and display user's progress for achieving one or more goals.

Communications network 18 couples and facilitates wireless or wire-line communication between computer devices 16, stress module 17, handheld devices 19, and servers 32. Communications network 18 may, for example, communicate Internet Protocol (IP) packets, Frame Relay frames, Asynchronous Transfer Mode (ATM) cells, voice, video, data, and other suitable information between network addresses. Communications network 18 may also communicate data via wireless communications, such as by Wireless Application Protocol (WAP) standard protocols, including 802.11, third-generation (3G) protocols (such as W-CDMA or CDMA 2000, for example), Bluetooth, or Global System for Mobile Communications (GSM) protocols, for example. Communications network 18 may include one or more local area networks (LANs), radio access networks (RANs), metropolitan area networks (MANs), wide area networks (WANs), interactive television networks, all or a portion of the global computer network known as the Internet, and/or any other communication system or systems at one or more locations.

A feature of the present invention includes a subscription model that may include users 12 paying to use web portal 40 and/or paying to use stress module 17. For example, server 32 can disable and/or enable certain functions and modes of stress module 17. Server 32 can configure all stress modules 17 of a group of users 12, such that all stress module 17 used by a particular business entity are configured with the same functionality. If user 12 is delinquent in subscription payments, stress module 17 may be disabled completely and access to web portal 40 may be blocked.

Software and/or hardware may reside in stress module 17 in order to achieve the teachings of collecting data, converting data, displaying data, and communicating data of the present invention. In one simplified example, stress module 17 is an algorithm that can be deployed in health station 22, or in computing devices 16, or in handheld devices 19. However, due to its flexibility, stress module 17 may alternatively be equipped with (or include) any suitable software, hardware, component, device, application specific integrated circuit (ASIC), processor, microprocessor, algorithm, read-only memory (ROM) element, random access memory (RAM) element, erasable programmable ROM (EPROM), electrically erasable programmable ROM (EEPROM, field-programmable gate array (FPGA), or any other suitable element or object that is operable to facilitate the operations thereof. Considerable flexibility is provided by the structure of stress module 17 in the context of communication system 10 and, accordingly, it should be construed as such.

Computer devices 16 may comprise computer systems that include appropriate input devices, output devices, mass storage media, processors, memory, or other components for receiving, processing, storing, and/or communicating information with other components of system 10. As used in this document, the term “computer” is intended to encompass a docking station (although USB connections may obviate the need for a docking station entirely), personal computer, workstation, kiosk, network computer, wireless data port, wireless telephone, personal digital assistant (PDA), cellular telephone, game console, one or more processors within these or other devices, or any other suitable processing device. It will be understood that any number of computer devices 16 may be coupled to other computer devices 16 or communications network 18. Computer devices 16 or handheld devices 19 are generally operated by users 12 to access the interactive community.

Servers 32 are generally operable to provide an interface between users 12 and web portal 40. One or more servers 32 may be web application servers or simple processors operable to allow users 12 to participate with web portal 40 via the communications network 18 using a standard user interface language such as, for example, the Hypertext Markup Language (HTML). In some embodiments, one or more servers 32 may be physically distributed such that each server 32, or multiple instances of each server 32, may be located in a different physical location geographically remote from each other. In other embodiments, one or more servers 32 may be combined and/or integral to each other. One or more servers 32 may be implemented using a general-purpose personal computer (PC), a Macintosh, a workstation, a UNIX-based computer, a server computer, a kiosk, or any other suitable processing device. Server 32 may include a processor to convert data and utilize algorithms. For example, server 32 may apply an algorithm to convert distance traveled into calories burned by utilizing data from the memory like a user's height, weight, and sex.

In some embodiments, servers 32 are operable to provide security and/or authentication of users 12 or other persons or entities attempting to access web portal 40. For example, servers 32 may essentially provide a firewall for entities attempting to access web portal 40. In addition, servers 32 may be operable to translate one or more data protocols used by web portal 40 with one or more protocols used by applications hosted by one or more computer devices 16 or handheld devices 19.

In particular embodiments, one or more servers 32 are web application servers operable to communicate dynamically updated information to particular computer devices 16 via communications network 18 including the identity of user 12. For example, one or more servers 32 may communicate updated information on web portal 40 to particular computer devices 16 or handheld devices 19 via communications network 18.

Servers 32 may further comprise a memory that may be accessed or otherwise utilized by one or more components of interactive community. The memory may take the form of volatile or non-volatile memory including, without limitation, magnetic media, optical media, random access memory (RAM), read-only memory (ROM), removable media, or any other suitable local or remote memory component. In general, the server memory may store various data including a user's account information, a user's goals, a user's activity data, and a population's activity data.

Web portal 40 comprises one or more web sites, hardware, and software that provide users of the web with the ability to search for information on the web including information in the web portal 40, documents, media, or other resources coupled to the web. The web sites on web portal 40 may include user's websites and informational websites. Web portal 40 provides a central location for users to get together with each other.

In accordance with the teachings of one configuration of the present invention (specifically, a health station model), communication system 10 achieves an effective way for end users to be rewarded for a successful completion of their assigned tasks in completing a stress test using stress module 17. This can be performed at a remote health station 22.

Taking a step back from the exact nuances of the present invention, note that virtually all companies have incentives (point systems, merchandising systems, etc.) to address rising healthcare costs. Stress is part of this healthcare equation. One distinction to be drawn between those systems and the proposed architecture is that the present invention can be used on either a short-term or a long-term basis. The parent company or business (that employs the participant) is empowered to set timeframes for the participant: those time frames being due in the near-term or over an extended period. In turn, the participant is rewarded immediately or systematically over a longer period of time.

For example, a participant may be assigned a near-term goal of performing a certain activity (e.g., focused relaxation) three times per week, while his long-term goal may be to lose 40 lbs. Progressively, health station 22 can monitor his achievements in both endeavors. Furthermore, as an incentive, health station 22 can deliver a reward to a participant as an enticement for continued and future compliance. Intuitively, the end user begins to understand that he will be rewarded quickly for his accomplishments.

Additionally, Kinetic Activity Monitor (KAM) points (which are further detailed below) can be earned by the participant. These points (or other types of merit points) can be used as currency such that an end user could earn points during the week and then convert those points to a reward such as those outlined in previous filings by Applicant and which are incorporated by reference below.

The present invention does utilize some technology previously applied for by Applicant. Note that the following related cases, which address aspects of the aforementioned website, healthcare methodologies, potential rewards, and features of end user devices, are hereby incorporated by reference: Accelerometer for Data Collection and Communication, Ser. No. 10/996,500, filed Nov. 23, 2004; System and Method for Implementing an Interactive Online Community Utilizing an Activity Monitor, Ser. No. 11/625,063, filed Jan. 19, 2007; System and Method for Population Health Management Data Collection and Communication: Ser. No. 10/915,852, filed Aug. 11, 2004; System and Method fox Codifying Risk Factors By a Healthcare Individual at a Remote Location: Ser. No. 11/760,457, filed Jun. 8, 2007; Music and Accelerometer Combination Device for Collecting, Converting, Displaying, and Communicating Data: Ser. No. ______, filed September 2007; and System and method for Incentivizing a Healthcare Individual Through Music Distribution: Ser. No. ______, filed Feb. 21, 2008.

It should be noted that the internal structure of the system of FIG. 1 is versatile and can be readily changed, modified, rearranged, or reconfigured in order to achieve its intended operations or additional operations. Additionally, any of the items within FIGS. 1-4 may be combined, where appropriate, or replaced with other functional elements that are operable to achieve any of the operations described herein.

System 10 may utilize communication protocols and technologies to provide communication sessions. Examples of communication protocols and technologies include those set by the Institute of Electrical and Electronics Engineers, Inc. (IEEE) standards, the International Telecommunications Union (ITU-T) standards, the European Telecommunications Standards Institute (ETSI) standards, the Internet Engineering Task Force (IETF) standards (for example, IP such as mobile IP), or other standards.

According to the illustrated embodiment, health station 22 represents any suitable device operable to collect biometric data from an end user, provide visual and audio communication session between the end user and a healthcare professional, and exchange information between the end user and a healthcare individual in essentially real-time. In this example, a finger strap is also provided at health station 22. Health station 22 may represent a computer, server, or data processing system, depending on context and applicable tasks. In the current embodiment, health station 22 is located within an entity (e.g., a corporate office). Health station 22 can include a memory for storing a participant's identification data and health data.

In one example, health station 22 may be constructed from wood in the shape of a bench seat, including a monitor, a telephone, a video camera, and a weight scale, such that the weight scale is positioned under the seat so that a participant can measure weight while sitting. In another embodiment, health station 22 may be constructed from metal in the shape of a rectangular box, including a monitor, built in speaker, and built in microphone. Participants can interact with health station 22 to receive an intervention plan from a healthcare individual via a video session. Health station can schedule an appointment for individual to connect to healthcare individual via a live video session. Alternatively, health station can show a pre-recorded video session to communicate between a participant and a healthcare individual.

Health station 22 can capture a multitude of data. For example, health station 22 can capture a participant's name, stress results, risk factors, health risk appraisal data, biometric data, utilization data, medical records, health insurance enrollment data, and any other relevant data. Health station 22 can save data associated with each participant on a remote server such that health station 22 will have a participant's information for subsequent visits. Health station 22, including biometric collection devices and electronic intervention modules, can be customized and configurable by authorized individuals, such as healthcare individuals. For example, entity ABC can configure their health station 22 so that activity monitors can connect to health station. Other architectures and components of health station 22 may be used without departing from the scope of this disclosure.

In an alternative embodiment, a participant can communicate with a healthcare individual to receive acute care or participate in an intervention plan by using a computing device with a display, such as a desktop computer, laptop, PDA, cell phone, etc. For example, healthcare coverage from employer may also cover spouses of employees. A spouse of employee can use their computer at home to communicate with a healthcare individual over a real-time, live video connection.

FIGS. 2-4 are example screen shots that illustrate how the algorithm can be used in conjunction with a web-site. In this example, the user is required to log-in before accessing stress module 17. Once the end user has been authenticated (for example through a log-in/unique identifier, as outlined herein), he may then strap on the finger strap (or this could be replaced by other various heart-rate variability measuring devices, for example a connection to the ear lobe, the wrist, the chest, the neck, etc.). All such monitoring tools are clearly within the broad scope of the present invention. Once the strap (in this case, a simple stress sensor) is secured, the user may elect to calm himself for a moment, and then proceed with the test. FIG. 2 further illustrates the previously recorded readings for this particular user, as well as some general guidance and instructions that lead the user through the stress process.

The algorithm in this example is running a simple process that takes about two (2) minutes to perform. The actual test is only 100 seconds long (the first 25-30 seconds are reserved for simple calibration) and during the test (every five (5) seconds in this example), the end user is told whether he is in a High, Medium, or Low level. In this example, the High, Medium, and Low levels are associated with a coherence attribute. Also, in this example, there are prominently displayed colors associated with High, Medium, and Low stress levels, which are Red, Blue, and Green respectively. While the test is run, an end user is given one full point if the end user is in the low range, one half of one point if the end user is in the medium range, and one quarter of one full point if the end user is in the high range. These points will be aggregated to deliver a score to the end user.

Note that this example is merely being used for illustrative purposes. The test could be longer than 100 seconds, or shorter. In essence, the test is broken up into intervals, which can be fashioned in various ways (e.g., 5 seconds, 1 millisecond, 1 minute, etc.). Additionally, a value can be assigned to each zone, where a strong coherence would offer a higher value than a lower coherence. Alternatively, this could be reversed where the objective is to achieve a lower ultimate score, whereby a low coherence (poor) would yield more points and the aggregate score would be higher.

The actual strap around the finger is set (in this example configuration) to take nineteen (19) readings per second. There are variations within the second intervals that are used to aggregate the amount of points the end user accumulates. In this example, there are twenty, five-second intervals. The end user is being scored at each interval based on the HRV statistics that are being gathered.

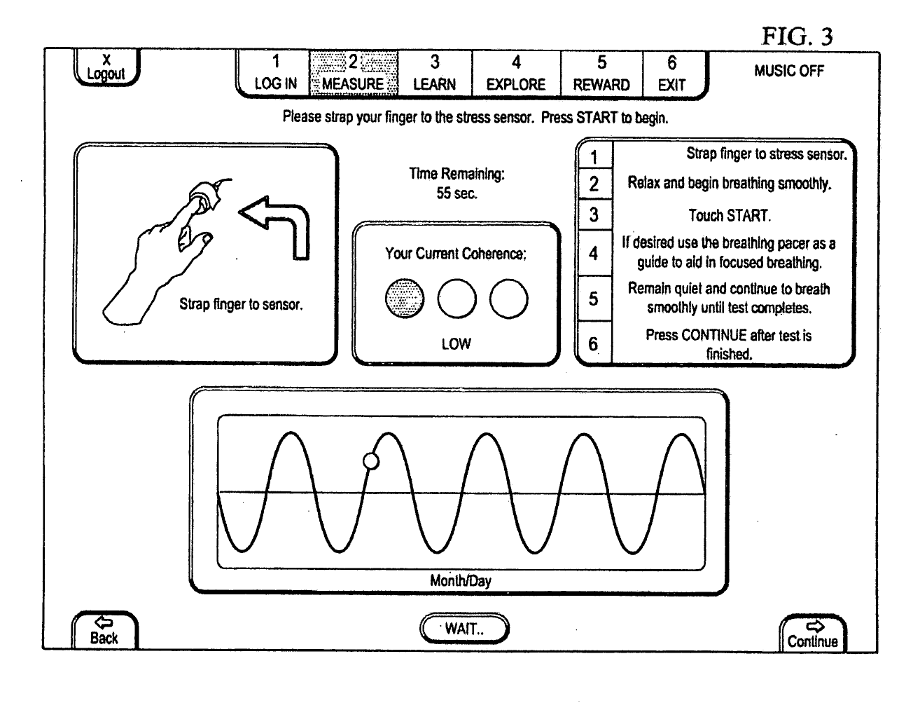

FIG. 3 illustrates the first reading in this example, and the end user here has a low stress level. A sinusoidal wave is also illustrated and it can mark progress and biorhythms detected during the testing. Note that sounds can be provided for each level (or each transitioning between stress levels) such that the end user is signaled about which level he is occupying. Similarly, messaging may be provided that either encourages or helps to motivate the user to relax while the testing occurs. Part of the experience in using this stress module is to learn to control biometric reactions in real-time. The presence of stress detracts from concentration levels, inhibits creativity, etc. and there is a current training movement for executives, athletes, etc. to be cognizant of their stress levels. Thus, indirectly the stress module is teaching its end users to control their levels of stress, to center themselves, and to manage their biometric responses: particularly so in times of high anxiety and stress. Another aspect of the present invention involves offering a test that is deliberately more challenging/difficult for the end user. This could further train the end user to manage his own stress levels during mentally demanding scenarios. In essence, scores are tied to levels such that a score of 81 earned at Level 1 may not be earned at Level 2, where the same person scores a 63. These more difficult levels provide a training exercise much like that associated with a conventional treadmill. In some cases, Levels may be varied for users who have grown accustomed to a certain Level: meaning they have mastered their existing Level and, thus, need a more challenging environment.

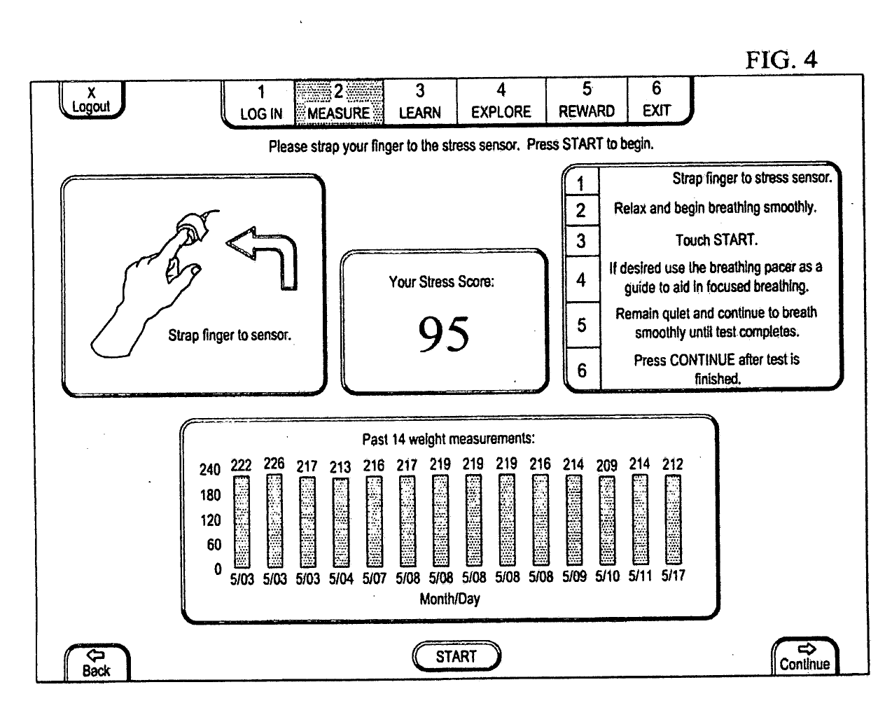

FIG. 4 illustrates that the test has been completed. The end user will leave the application with a number (potentially decimal-based) for that particular testing scenario. There will be ranges for indicating that levels are acceptable, low, or high. These could be provided on the web-site, on a chart, on a card, at health station 22, or at any other suitable place. In this instance, the end user has excellent stress levels and has scored a 95. This information will be stored and/or transmitted to any suitable destination (either wirelessly or stored directly on the device that assisted in gathering this information). In this instance, the end user has excellent stress levels and has scored a 95.

Once the results of the stress test have been obtained, any various areas may be accessed to further enlighten the end user (or address questions that he may have). In the depiction of FIG. 4, there are areas for learning, exploring, and rewarding participants who engaged in the test. These subsequent activities may ultimately yield a more comprehensive experience for the end user, as he manages his stress levels.

For example, a number of modules that address specific problems identified as relevant stress factors could be provided. Successful completion of the module(s) would result in a reward (monetary or otherwise). The specific modules may include any exercise or task to be completed by the targeted participants. The modules are designed after identifying the relevant factors associated with the target population, as it pertains to stress. As used herein, the term “module” includes any task to be completed by the targeted participant in the context of an intervention plan. Completion of the modules can earn merit points of some kind (e.g., KAM points) that can be used or swapped for other items.

The modules can be selected intelligently from health station 22, or based on a participant's health data, such that modules are displayed to a participant via health station 22. Alternatively, modules can be created by healthcare individuals based on a participant's health data such that a healthcare individual explains module via live video feed to a participant at health station 22. In the context of an intervention plan, the modules are designed after analyzing the health data and identifying relevant risk factors associated with the target population. Hence, the identified stress levels can be used as the basis for configuring the modules, which can be interactive. Considerable time and effort may be expended in designing the precise modules that will yield the most beneficial results for the target group and, thereby, alleviate the healthcare costs for a given population of participants.

Alternatively, a healthcare individual can immediately develop a module customized to a participant based on the participant's health data transmitted from health station 22. Thus, the modules in the context of an intervention plan are designed to modify factors and related healthcare expenses for a given participant or group. The modules associated with an intervention plan may also achieve a reduction in healthcare expenses by modifying the choices of the participant so that the participant chooses new behaviors or abandons old behaviors that are costly.

Therefore, a stress module associated with an intervention plan could include virtually any action, exercise, or assignment that may affect a participant's beliefs, feelings, thoughts, or behaviors. This is inclusive of a participant refraining from doing some action or intentionally not participating in certain endeavors. There could be a series of successive modules to be completed by a participant in a particular order, or the modules could be completed in a random fashion. A module associated with an intervention plan is tailored specifically for a participant or a group of participants and, therefore, modules are considerably flexible and malleable. A module associated with an intervention plan may be completed during normal business hours (potentially under the supervision of an administrator), during non-business hours where the ‘honor system’ is employed, or anytime. Furthermore, an incentive program can be implemented, such that more participants will comply with intervention plans.

Note that the modules associated with the intervention plans can be primarily action or process-oriented, as opposed to information-oriented, so that their focus is on the facilitation of change in the participant. The modules can be designed to allow a participant to acquire skills and life applications of the learned information. The participant may be asked to respond affirmatively in order to address certain subject matter. In addition, a participant may be required to perform specific tasks. Rewards may then be given based on the performance of the modules by a participant, as he completes, applies, acquires, or participates in proscribed assignments within the modules.

A module associated with a stress intervention plan could include educational tools, such as a booklet, video, or computer program designed to address the illness, behavior, or issue presented by the target participant or group. For example, for stress management, a video could include information about proper diet (e.g. inclusive of caffeine restrictions), breathing exercises, time management, and sleeping suggestions. The booklet could include electronic fill-in the blank questions that quiz the participant on the lessons learned.

Modules associated with an intervention plan can also be related to physical exercises to be completed by the participants. An honor system may be employed for such a module or a participant may wear some type of activity monitor (e.g. a pedometer for tracking walking, a heart rate monitor for tracking other activities, etc.). In addition, a module associated with an intervention plan may include work completed using access terminal, health station, and, potentially, monitored by an on-line administrator. A module associated with an intervention plan could also simply be the completion or achievement of a specific goal.

It is imperative to note that the modules identified above only offer one simple example of how follow-up information (following the stress test) can be delivered to the target group. The specific modules above may readily be replaced with any other suitable module that targets specific factors or character observations or the disease conditions of the individual. Moreover, modules could be completed in a specific manner (inclusive of timelines and deadlines) such that the expected result is achieved. Considerable flexibility is provided by these modules as they are tailored to meet the exact needs of the individuals in the target group. It can be appreciated that the module arrangements presented here are arbitrary, as they have been only used for purposes of teaching. Accordingly, any module configurations offered herein in this document should be construed as such: simply one example of the millions of possible combinations and arrangements that may be used.

It also is important to note that the stages and steps described above in the preceding FIGURES illustrate only some of the possible scenarios that may be executed by, or within, the present system. Some of these stages and/or steps may be deleted or removed where appropriate, or these stages and/or steps may be modified, enhanced, or changed considerably without departing from the scope of the present invention. In addition, a number of these operations have been described as being executed concurrently with, or in parallel to, one or more additional operations. However, the timing of these operations may be altered.

The preceding example flows have been offered for purposes of teaching and discussion. Substantial flexibility is provided by the tendered architecture in that any suitable arrangements, chronologies, configurations, and timing mechanisms may be provided without departing from the broad scope of the present invention. Accordingly, communications capabilities, data processing features and elements, suitable infrastructure, and any other appropriate software, hardware, or data storage objects may be included within health station to effectuate the tasks and operations of the elements and activities associated with executing compatibility functions.

Certain features of the invention have been described in detail with reference to particular embodiments in FIGS. 1-4, but it should be understood that various other changes, substitutions, and alterations may be made hereto without departing from the sphere and scope of the present invention. For example, although the preceding FIGURES have referenced a number of relevant health rise factors, any suitable characteristics or relevant parameters may be readily substituted for such elements and, similarly, benefit from the teachings of the present invention. These may be identified on a case-by-case basis, whereby a certain participant may present a health risk factor while another (with the same condition) may not. Thus, a statistical relevance may be identified for one group, but not another who appears to be similar. Additionally, different and unique intervention plans can be customized by healthcare individuals and/or servers.

What is claimed is:

1. A method, comprising:
interacting with an end user through a stress module;
processing heart rate variance data collected during a stress test, wherein the heart rate variance data may include a coherence attribute; and
transmitting the heart rate variance data over a network and to a specified location.

2. The method of claim 1, further comprising:
delivering a numerical score to the end user, and wherein the score signifies a stress level for the end user.

3. The method of claim 1, wherein the interacting is performed through a website, a health station, a handheld device, or a personal computer.

4. The method of claim 1, wherein the heart rate variance data is stored and sent to a first destination and then to a second destination over a network.

5. The method of claim 4, wherein the handheld device is a cellular phone, an I-Phone, a PDA, or a handheld stress test tool.

6. The method of claim 1, wherein the test includes designations for Low, Medium, and High stress levels, whereby each stress level includes a respective range of stress scores.

7. The method of claim 1, wherein the test includes sounds that are played for the end user, or messaging provided to the end user.

8. The method of claim 1, further comprising:
providing educational materials to the end user after the stress test.

9. The method of claim 1, wherein the stress test includes a color scheme that indicates levels of stress for the end user.

10. The method of claim 1, further comprising:
providing a health station as the remote location;
capturing stress data from the end user at the health station;
storing the stress data; and
analyzing the stress data to determine an intervention plan for the end user.

11. An apparatus, comprising:
a stress module interacting with an end user, wherein the stress module processes heart rate variance data collected during a stress test, the heart rate variance data including a coherence attribute, and wherein the stress module delivers a numerical score to the end user, wherein the score signifies a stress level for the end user.

12. The apparatus of claim 11, wherein the score is based on a plurality of time intervals associated with the test.

13. The apparatus of claim 11, wherein the interacting is performed through a website, a health station, a handheld device, or a personal computer.

14. The apparatus of claim 11, further comprising:
a memory that stores heart rate variance data, wherein the data is sent to a first destination and then to a second destination over a network.

15. The apparatus of claim 11, wherein the test includes designations for Low, Medium, and High stress levels, whereby each stress level includes a respective range of stress scores.

16. Software for processing data, the software being embodied in a component that includes a processor and a memory, the software when executed by the processor being operable to:
interact with an end user via a stress module; and
process heart rate variance data collected during a stress test, wherein the heart rate variance data includes a coherence attribute; and
deliver a score to the end user, wherein the score signifies a stress level for the end user.

17. The software of claim 16, wherein the score is based on a plurality of time intervals associated with the test.

18. The software of claim 16, wherein the interacting is performed through a website, a health station, or a personal computer.

19. The software of claim 16, wherein the interacting is performed through a handheld device, and wherein the handheld device is a cellular phone, an I-Phone, a PDA, or a handheld stress test tool.

20. The software of claim 16, wherein the test includes designations for Low, Medium, and High stress levels, whereby each stress level includes a respective range of stress scores, and wherein the test includes sounds that are played for the end user, and wherein the stress test includes a color scheme that indicates levels of stress for the end user.
